(19) United States
(12) Patent Application Publication
(10) Pub. No.: US 2024/0198121 A1
(43) Pub. Date: Jun. 20, 2024

(54) TOOL FOR A PLASMA MEDICAL TREATMENT DEVICE, AND CORRESPONDING DEVICE

(71) Applicants: SORBONN EUNIVERSITE, Paris (FR); ASSISTANCE PUBLIQUE - HOPITAUX DE PARIS, Paris (FR); CENTRE NATIONAL DE LARECHERCHE SCIENTIFIQUE, Paris (FR); INSTITUT NATIONAL DE LA SANTE ET DE LA RECHERCHE MEDICALE-I.N.S.E.R.M., Paris (FR); ECOLE POLYTECHNIQUE, PALAISEAU (FR)

(72) Inventors: Thierry DUFOUR, Paris (FR); Laura FOUASSIER, MAISONS-ALFORT (FR); Marine CAMUS, VERSAILLES (FR); Henri DECAUCHY, Paris (FR)

(73) Assignees: SORBONNE UNIVERSITE, Paris (FR); ASSISTANCE PUBLIQUE - HOPITAUX DE PARIS, Paris (FR); CENTRE NATIONAL DE LARECHERCHE SCIENTIFIQUE, Paris (FR); INSTITUT NATIONAL DE LA SANTE ET DE LA RECHERCHE MEDICALE-I.N.S.E.R.M., Paris (FR); ECOLE POLYTECHNIQUE, PALAISEAU (FR)

(21) Appl. No.: 18/287,328

(22) PCT Filed: Apr. 28, 2021
(86) PCT No.: PCT/FR2021/000041

§ 371 Date: Oct. 18, 2023

(51) Int. Cl. A61N 1/44

(52) U.S. Cl. CPC A61N 1/44

(57) ABSTRACT

A tool for generating a plasma, the tool having at least one instrument, the instrument having at least a power supply electrode, an internal dielectric screen, a counter electrode, and a sheath. A device that has such a tool.

The invention relates to a tool for a plasma medical treatment device.

Such a tool can also be used on a patient (human) or on an experimental model (animal).

Such a tool can be used in numerous branches of medicine such as, and in a non-limiting manner, in otolaryngology, pulmonology, gastroenterology (upper part and lower part of the digestive system and, for example, oesophagus, stomach, pancreas, large intestine, small intestine, duodenum, biliary tree, etc.), laparoscopy, gynaecology (obstetrics included), dermatology, orthopaedics, etc.

Such a tool can be used for numerous medical applications, such as, and in a non-limiting manner, oncology application (anti-tumour effect, for example), fluid and/or natural cavity and/or cell tissue and/or organ decontamination, etc., fighting against stenosis (in particular, bile ducts), fighting against atresia, assisting blood coagulation, surface chemical activation, coating, stimulation or regeneration of cell tissues and/or organs, chemical functionalisation, etc.

Such a tool can be implemented to apply a plasma directly on a given zone to be treated and/or indirectly via a solution (liquid and/or gaseous) applied to the zone to be treated, the solution being or having been treated by plasma beforehand.

The invention also relates to a device comprising such a tool.

### BACKGROUND OF THE INVENTION

Plasma is considered as a material state in the same way as liquid, solid and gas. This “fourth state” can be obtained by ionising a gas subjected to an electric field or also brought to a high temperature.

It has been observed that the action of a cold (or non-thermal) plasma on tumours such as cholangiocarcinoma makes it possible to reduce its volume and to induce the apoptosis of cancer cells.

With medicine focusing more and more on targeted treatments, prototypes have thus been developed, making it possible to generate plasma in long, flexible tubes. However, their insertion into an endoscope makes them dangerous. Indeed, the internal structure of a conventional endoscope has certain metal walls; walls which can therefore conduct the electrical current and thus electrify both the patient (or experimental model) and the operator. In summary, the current prototypes do not make it possible to generate a plasma in a catheter-type tool, itself inserted into an endoscope, while being safe both for the patient (or experimental model) and the operator.

### Aim of the Invention

An aim of the invention is to propose a tool for a medical treatment device which is totally safe relative to a patient (or an experimental model) and a practitioner.

An aim of the invention is also to propose a medical treatment device incorporating such a tool.

### SUMMARY OF THE INVENTION

In view of achieving this aim, a tool for generating a plasma is proposed, the tool comprising at least one instrument.

According to the invention, the instrument comprises at least, from the inside to the outside:
a power supply electrode,
an internal dielectric screen,
a sheath,
the instrument further comprising at least one counter electrode arranged externally to the internal dielectric screen.

The inventors have been able to observe that the instrument thus described enabled a good isolation of the power supply electrode relative to an external environment which made it possible to protect a patient (or experimental model) treated by such a tool, as well as a practitioner handling the tool.

Optionally, the power supply electrode is a metal wire.

Optionally, the power supply electrode is polarised and the counter electrode is a grounding electrode.

Optionally, the instrument comprises at least one additional layer.

Optionally, the additional layer is chosen from among:
an external dielectric screen, and/or
a spacer layer.

It is retained that the external dielectric screen is called “external” opposed to the other internal dielectric screen which is always arranged more at the centre of the instrument.

Optionally, the additional layer is a spacer layer which is arranged on the internal dielectric screen or on the counter electrode.

Optionally, the instrument comprises two additional layers, namely an external dielectric screen and a spacer layer, the spacer layer being arranged between the internal dielectric screen and the sheath.

Optionally, the spacer layer is composed of a plurality of spacer rings which are independent from one another.

Preferably, the internal dielectric screen covers a distal end of the power supply electrode.

Optionally, there is at least one cap arranged at the distal end of the instrument to guide in service a passage of the plasma from the inside of the instrument to the outside of the instrument.

Optionally, the cap is an integral part of the counter electrode, thus forming the distal end of said counter electrode.

Optionally, the cap has at least one hydrophobic surface zone.

Optionally, at least the distal end of the counter electrode is shaped in a cylinder.

Optionally, the tool comprises a needle arranged on a distal part of the tool.

The tool can thus be used for a percutaneous treatment, by directly introducing its distal part provided with a needle into the body of the patient (or experimental model) which—de facto—does not require to resort to an endoscope.

The invention also relates to a medical treatment device comprising an applicator, wherein at least one conduit is provided, the tool such as mentioned above being arranged, such that at least its instrument extends into said conduit.

Advantageously, the tool can be implanted applicators which are currently on the market.

The invention also relates to a medical treatment method implemented using a tool such as mentioned above, comprising the steps of bringing the tool close to a zone to be treated and of generating a plasma by the tool, in order to subject said zone to be treated to said plasma.

Other features and advantages of the invention will emerge upon reading the description below of particular, non-limiting embodiments of the invention.

Below, and unless indicated otherwise, the intervals indicated must be understood as closed terminals.

### BRIEF DESCRIPTION OF THE DRAWINGS

The invention will be best understood in the light of the description below in reference to the accompanying figures among which:

FIG. 1 is a schematic view of a device according to a particular embodiment of the invention;

FIG. 2a is a perspective view of a medical tool according to a first embodiment of the invention associated with the device represented in FIG. 1,

FIG. 2b is a radial, cross-sectional view of the tool represented in FIG. 2a,

FIG. 2c is a cross-sectional view, along a cross-sectional plane parallel to an axial, cross-sectional plane, of the tool represented in FIG. 2a,

FIG. 3 represents possible variants of the tool illustrated in FIG. 2a,

FIG. 4a is a perspective view of a medical tool according to a second embodiment of the invention associated with the device represented in FIG. 1,

FIG. 4b is a radial, cross-sectional view of the tool represented in FIG. 4a,

FIG. 5 represents possible variants of the tool illustrated in FIG. 4a,

FIG. 6 represents different options of shapings of a dielectric screen of a medical tool associated with the device represented in FIG. 1.

### DETAILED DESCRIPTION OF THE INVENTION

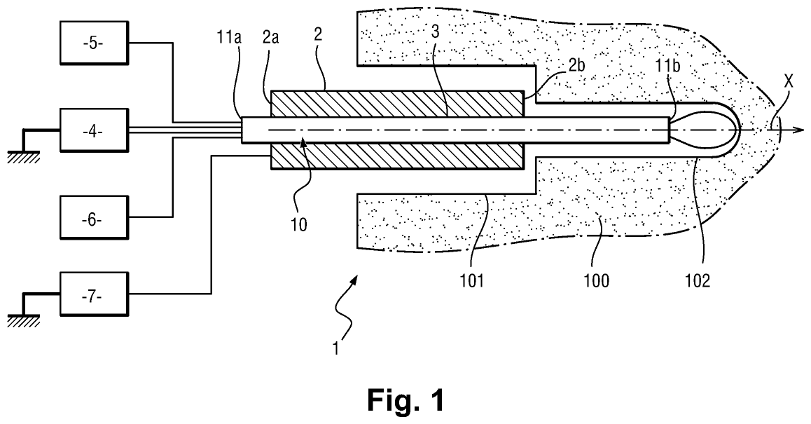

In reference to FIG. 1, the medical treatment device 1 according to a particular embodiment of the invention comprises an applicator which is an endoscope 2, in this case.

The endoscope 2 comprises at least one main conduit called working conduit passing through it throughout between a proximal end 2a of the endoscope intended to be arranged outside of a body 100 of a patient and a distal end 2b of the endoscope intended to be arranged in the body 100 of the patient in the proximity of a zone to be treated.

Below, the term “distal end” must be understood as being the end arranged on the distal end side 2b of the endoscope and “proximal end” as being the end arranged on the proximal end side 2a of the endoscope.

The endoscope 2 can be introduced into the patient via a natural or artificial cavity 101 of the patient.

In a manner known per se, the endoscope 2 is sufficiently flexible to be able to be deformed in order to follow, if needed, the natural path of the cavity 101 or of a channel 102 extending said cavity, and wherein it circulates (bile duct, digestive tube, etc.).

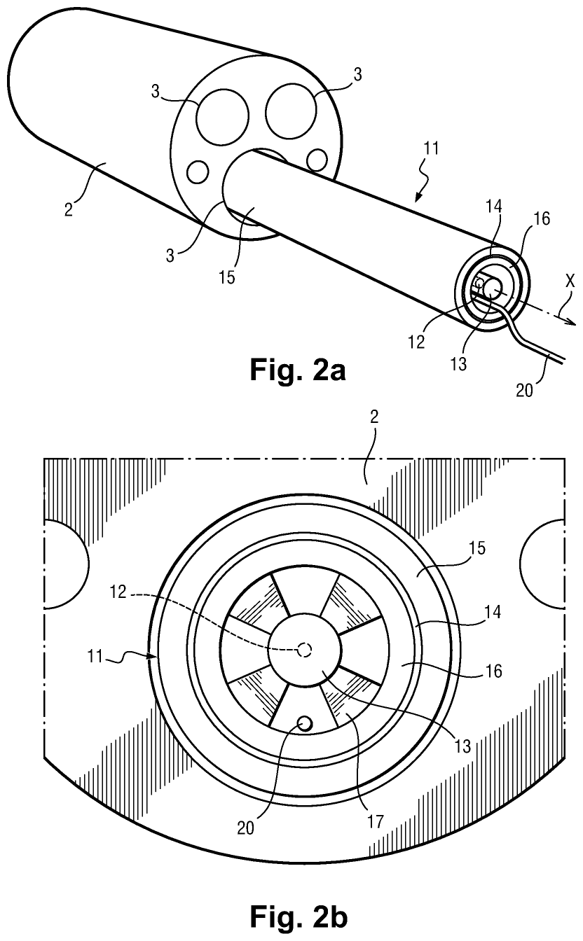

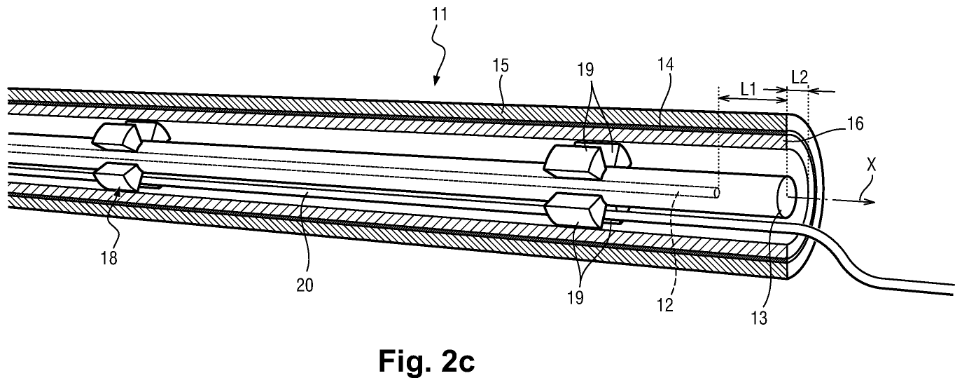

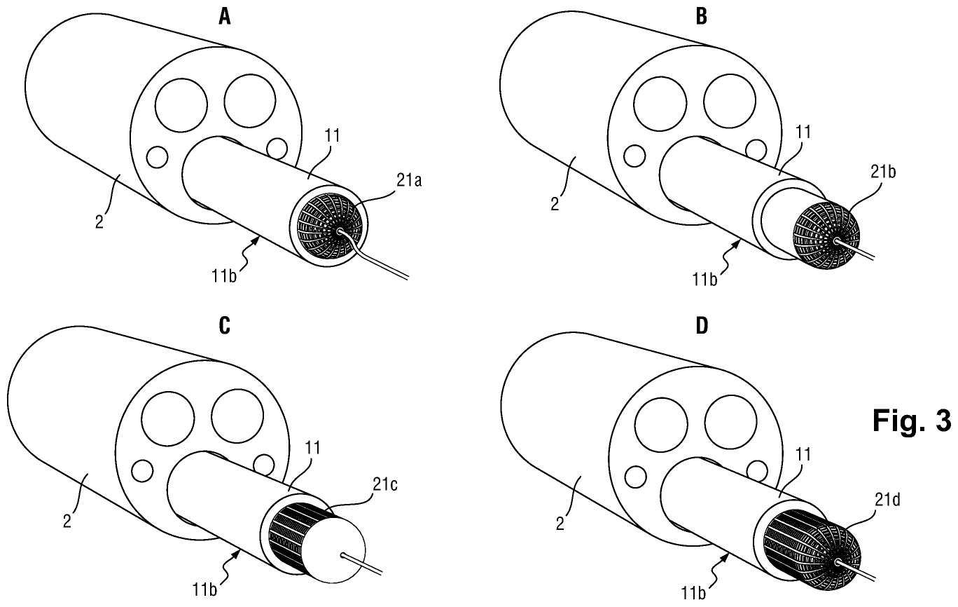

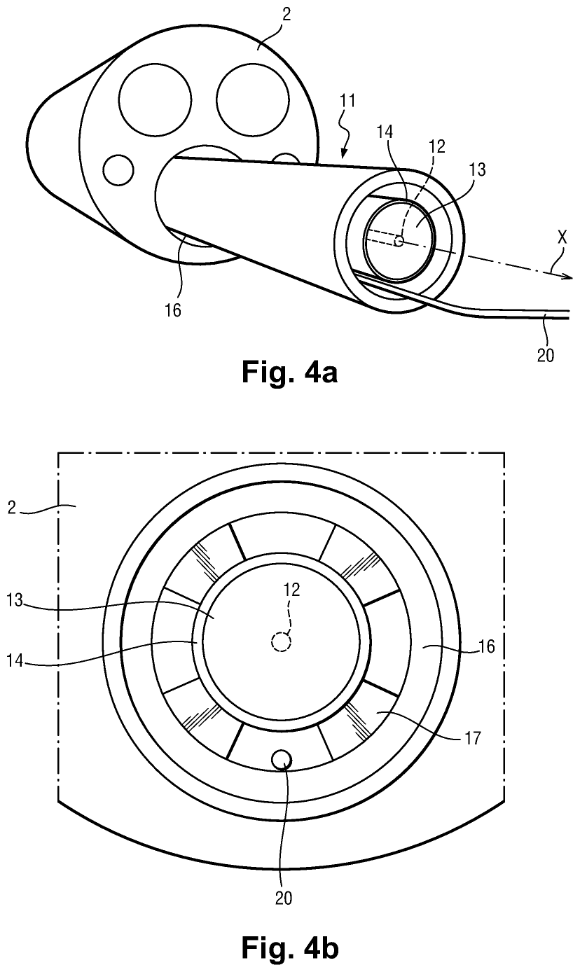

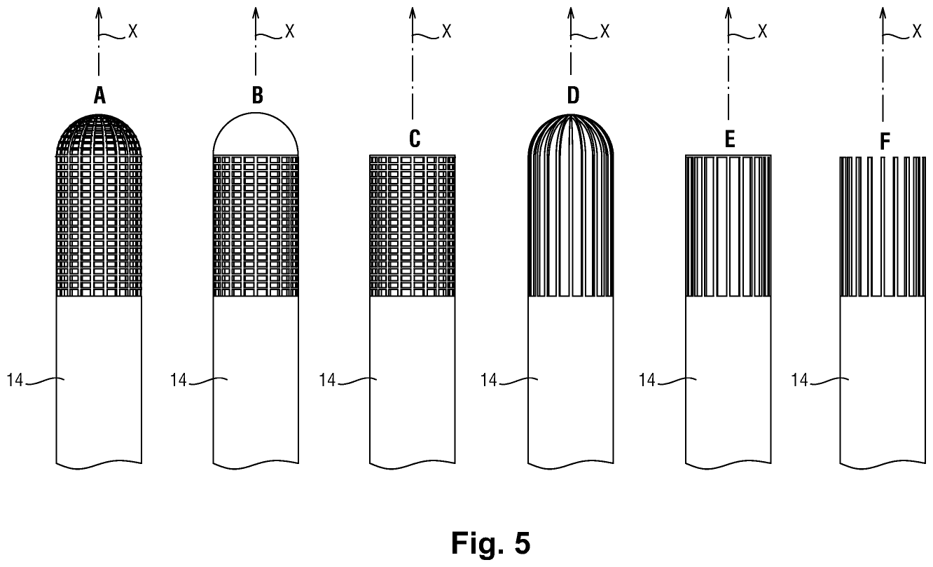

In the present case, and as can be better seen in FIGS. 2a and 4a, the endoscope 2 comprises three main conduits and two secondary conduits.

Moreover, the device 1 comprises a tool 10 capable of generating a plasma, the tool 10 comprising at least one instrument 11 which is arranged in one of the three main conduits 3 of the endoscope 2.

Said conduit 3 has a diameter, for example, of between 1 and 66 millimetres, and for example, between 2 and 10 millimetres, and for example, between 4 and 4.5 millimetres. Optionally, the conduit 3 has a diameter of 4.2 millimetres (being understood that this diameter must be necessarily sufficiently large such that the instrument 11 can be introduced there and therefore necessarily greater than the external diameter of the instrument 11—which will be that of the sheath or of the external dielectric screen of the instrument 11 in the embodiments described below).

In particular, the instrument 11 is not contiguous (in any case, not continuously over all of its length) to the conduit 3.

The tool 10 comprises one single instrument 11, in this case. The tool 10 is composed only of said instrument 11, in this case.

The instrument 11 extends coaxially with respect to the conduit 3 of the endoscope 2 in which it is arranged when the endoscope 2 (and therefore the instrument 11) extends in a rectilinear direction. Naturally, the instrument 11 is also sufficiently flexible, in order to be able to be deformed to follow the movement of the associated endoscope 2.

The instrument 11 is a tubular instrument.

The instrument 11 moreover extends axially in a general direction X.

The instrument 11 extends more specifically, in this case, such that at least one of its axial ends exceeds the endoscope 2. The tubular instrument 11 extends more specifically, in this case, such that its two axial ends protruding from the endoscope 2.

The distal end 11b of the instrument 11 thus extends outside of the endoscope 2 (distal end side 2b of the endoscope 2) and the proximal end 11a of the instrument 11 extends outside of the endoscope (proximal end side 2a of the endoscope 2).

The device 1 moreover comprises a system for generating a plasma which comprises at least one gas supply source 4 and at least one electrical energy supply source 5, each of said sources being connected to the tool 10.

The gas supply source 4 has, for example, the following parameters:
use of a carrier gas from among helium, air, argon, neon, etc., and/or
use of a carrier gas at a flow rate of between 0 and 10 litres per minute (according to the standard temperature and pressure conditions taken at 25 degrees Celsius and 1 bar—CSTP) and preferably of between 0.01 and 1 litre per minute, and which is preferably 0.1 litre per minute, and/or
use of one or more secondary gases chosen from among dihydrogen, dinitrogen, dioxygen, carbon dioxide, methane, and/or
use of at least one secondary gas at a flow rate of between 0 and 10 litres per minute (in CSTP) and preferably of between 0.01 and 1 litre per minute, and which is preferably 0.1 litre per minute, and/or
use of a continuous, static, turbulent flow system, etc.

The electrical energy supply source 5 has, for example, the following parameters:
use of a direct, alternating (square, sinusoidal, triangular, saw teeth shape, etc.), pulse voltage, etc., and/or
use of a voltage of a magnitude of between 12 volts and 12 kilovolts, and preferably of between 1 kilovolt and 10 kilovolts, and preferably of between 3 kilovolts and 6 kilovolts, and/or
use of a voltage of a frequency of between 0 (not included) and 27.12 megahertz and preferably of between 100 hertz and 10 kilohertz, and which is preferably of 1 kilohertz.

The device 1 moreover comprises a residual gas discharge system 6 not transformed into plasma and/or generated plasma present in the tool 10 and/or the instrument 11, the residual gas discharge system also being connected to the tool 10.

The device 1 can naturally comprise one or more other additional elements, such as, for example, a second electrical energy supply source 7, this time connected to the endoscope 2, or also a device for microfluidically controlling the flow rate of the carrier gas and/or secondary gas injected into the instrument 11.

The device 1 is advantageously designed to be able to operate according to several modes.

In a first mode, a gas is injected into the instrument 11: the plasma generated in the instrument 11 thus tends to be propagated beyond the distal end 11b of the instrument 11 in the form of a plume, of which it is possible to adapt the dimensions by modifying, for example, the flow rate of the carrier gas and/or the distance between the distal end of the instrument and the zone to be treated.

In a second mode, no gas is injected into the instrument. The plasma is thus generated directly in the gaseous mixture natively present in the instrument 11 generally around the internal dielectric screen of the latter.

In a third mode, a liquid and/or steam solution (for example, a physiological medium, a pharmacological medicine, etc.) is injected into the instrument 11. The solution is thus activated and/or treated by the plasma generated in the instrument 11 all throughout the progression of the liquid solution in the instrument 11 before reaching the zone to be treated.

In reference to FIGS. 2a, 2b and 2c, a tool 10 according to a first particular embodiment of the invention will now be described.

The instrument 11 of said tool 10 comprises, from the inside to the outside, at least four layers:
a power supply electrode 12,
an internal dielectric screen 13,
a sheath 16,
a counter electrode 14.

The power supply electrode 12 is therefore a polarised electrode. The power supply electrode 12 is made of an electrically conductive material and, for example, made of a metal by, for example, copper- and/or aluminium-based.

Preferably, the power supply electrode 12 is a single wire. The power supply electrode 12 is thus very structurally simple.

The external diameter of the power supply electrode 12 is, for example, of between 0.01 and 10 millimetres and, preferably, of between 0.01 and 5 millimetres, and for example, of between 0.05 and 0.35 millimetre, and for example, of between 0.1 and 0.3 millimetre.

It is noted that the power supply electrode 12 is directly connected to the electrical energy supply source 4. Consequently, its electrical potential is not floating and only depends on the electrical features of the electrical energy supply source 4 which are, moreover, known and fully controlled. Consequently, the power supply electrode 12 is an electrode, the value of which is always known (and therefore, non-floating).

Thus, a risk that the plasma transits towards a thermal arc system is limited. The instrument is therefore very safe to use for the practitioner, as it is for the patient. The internal dielectric screen 13 is, for example:
shaped in a tube which is closed at its two axial ends, or
a coating directly contiguous to the power supply electrode 12, the coating thus also covering the axial ends of the power supply electrode 12 (the internal dielectric screen 13 thus not having a specific shape, as it is not contiguous to the power supply electrode 12).

In this way, the power supply electrode 12 is fully arranged inside the internal dielectric screen 13: the plasma cannot therefore come into contact with said power supply electrode 12 in the same way as the zone to be treated or the immediate environment of said zone (tissue, biological fluid, etc.).

Thus, a risk that the plasma transits towards a thermal arc system is limited.

The internal dielectric screen 13 is naturally made of a dielectric material.

The internal dielectric screen 13 is, for example, made of a natural or artificial rubber. The internal dielectric screen 13 is, for example, made of plastic material (polyvinylidene fluoride, polytetrafluoroethylene, perfluoroalkoxy, etc.).

The external diameter of the internal dielectric screen 13 is, for example, of between 0.01 and 20 millimetre, and for example, of between 0.5 and 3 millimetres, and for example, of between 1.0 and 1.1 millimetre (being understood that this diameter is moreover necessarily greater than that of the power supply electrode 12).

The sheath 16 is shaped in a tube, open at its two axial ends.

Thus, the sheath 16 forms a carrier structure of the instrument 11.

The sheath 16 is preferably made of a dielectric material.

The sheath 16 is, for example, made of a natural or artificial rubber. The sheath 16 is, for example, made of plastic material (polyvinylidene fluoride, polytetrafluoroethylene, perfluoroalkoxy, etc.).

The external diameter of the sheath 16 is, for example, of between 0.4 and 46 millimetres, and for example, of between 1.2 and 4 millimetres, and for example, of between 2.0 and 2.5 millimetres (being understood that this diameter is necessarily greater than that of the lower layer which is directly adjacent to it).

The counter electrode 14 is preferably a grounding electrode. In a variant, the counter electrode 14 is a floating electrode.

The counter electrode 14 is, for example, made of metal. The counter electrode 14 is optionally copper- and/or aluminium-based, or made of thereof.

The external diameter of the counter electrode 14 is, for example, of between 0.6 and 56 millimetres, and for example, of between 1.3 and 6 millimetres and for example, of between 2.2 and 2.7 millimetres (being understood that this diameter is moreover necessarily greater than that of the lower layer which is directly adjacent to it).

Preferably, the instrument 11 comprises at least one additional layer arranged above the internal dielectric screen 13.

The additional layer is, for example, chosen from among:
an external dielectric screen 15 and/or
a spacer layer 17.

In the present case, the instrument 11 comprises the two abovementioned additional layers.

The spacer layer 17 is, in this case, arranged between the internal dielectric screen 13 and the sheath 16.

The spacer layer 17 is, for example, shaped such that the radial distance separating the internal dielectric screen 13 of the sheath 16 is preferably of between 10 micrometres and 10 millimetres and is preferably of between 0.1 and 1 millimetre and is preferably of between 0.4 and 0.6 millimetre, and is for example of 0.5 or 0.45 millimetre.

The external diameter of the spacer layer 17 is, for example, of between 0.3 and 40 millimetres, and for example of between 1 and 3.5 millimetres and for example of between 1.6 and 1.9 millimetre (being understood that this diameter is moreover greater than that of the lower layer which is directly adjacent to it).

The spacer layer 17 is, in this case, composed of a plurality of spacer rings 18 which are independent from one another. However, each of these rings 18 enables the same gap between the internal dielectric screen 13 and the sheath 16, such that they together form a general spacer layer. FIG. 2c makes it possible to see said spacer layer 17 better.

Preferably, the rings 18 are arranged at regular intervals around the internal dielectric screen 13 in the direction X. The distance (in the direction X) separating two consecutive rings 18 is therefore the same between two pairs of different rings.

The distance separating two consecutive rings 18 (in the direction X) is, for example, less than 15 centimetres and preferably less than 10 centimetres.

At least one of the rings 18 is made of a dielectric material. At least one of the rings 18 is, for example, made of a natural or artificial rubber. At least one of the rings 18 is, for example, made of plastic material (polyvinylidene fluoride, polytetrafluoroethylene, perfluoroalkoxy, etc.).

Preferably, the different rings 18 are identical to one another, such that the description below of one of the rings 18 is also applicable to the other rings 18.

The ring 18 is, in this case, composed of at least two elements which are independent from one another. In the present case, the ring 18 is composed of four elements, which are independent from one another.

The ring 18 is mainly shaped in a ring, each element forming a segment 19 of this ring. The four elements are preferably distributed circumferentially regularly around the internal dielectric screen 13. Each element is arranged substantially at 90 degrees from each of the two elements of the same ring 18 surrounding it.

Preferably, each ring 18 is oriented in the same way vis-à-vis the internal dielectric screen 13. Consequently, a segment of a ring 18 is necessarily aligned (in the direction X) with another segment of each of the other rings 18.

Each element being identical, the description below of one of the segments 19 is also applicable to the other segments.

The length of the segment 19 (in the direction X) is preferably of between 0.2 millimetre and 100 millimetres, and preferably of between 2 and 20 millimetres and preferably of between 3 and 6 millimetres, and is preferably of 5 millimetres.

Moreover, the thickness of the segment 19 (in a radial direction) is preferably of between 10 micrometres and 10 millimetres, and preferably of between 0.1 and 1 millimetre, and is preferably of 0.5 millimetre.

Each segment 19 can extend to have an external periphery forming an angle sector between 35 and 50 degrees and, for example, be of 45 degrees.

The external dielectric screen 15 is, for example, shaped in a tube which is open at its two axial ends or corresponds to a coating layer directly affixed to the counter electrode 14 by preferably covering the axial ends of said counter electrode 14 (the external dielectric screen 15 thus not having a specific shape as it is not affixed to the counter electrode 14).

The external dielectric screen 15 is naturally made of a dielectric material.

The external dielectric screen 15 is, for example, made of a natural or artificial rubber. The external dielectric screen 15 is, for example, made of plastic material (polyvinylidene fluoride, polytetrafluoroethylene, perfluoroalkoxy, etc.).

The external dielectric screen 15 is preferably transparent.

The external diameter of the external dielectric screen 15 is, for example, of between 0.8 and 66 millimetres, and for example, of between 2 and 10 millimetres, and for example, of between 3.2 and 4 millimetres (being understood that this diameter is moreover necessarily greater than that of the counter electrode 14).

Optionally, when at least the distal end 11b of the instrument 11 extends rectilinearly:
the distance L1 in the direction X separating the distal face of the power supply electrode 12 from that of the internal dielectric screen 13 is between 0 (the value 0 being excluded from the interval) and 20 millimetres, and preferably is between 0.5 and 5 millimetres, and preferably between 0.8 and 3 millimetres, and is for example 1 millimetre or 2.5 millimetres,
and/or
the distance L2 in the direction X separating the distal face of the internal dielectric screen 13 from the distal end of the sheath 16 is between 0 millimetres and 1 metre, and preferably is between 2 and 10 millimetres, and preferably between 4 and 7 millimetres, and is for example, 5 millimetres.

The distance L2 in the direction X separating the distal face of the internal dielectric screen 13 from that of the sheath 16 can be equal to zero, for example and in a non-limiting manner, for dermatological applications.

The “power supply electrode 12 and internal dielectric screen 13” assembly not being integral in the present case of the counter electrode 14 and of the sheath 16, the distance L2 can easily be modified during an intervention by the practitioner, as needed.

The counter electrode 14 is shaped in a tube which is open at its two axial ends, or corresponds to a coating layer directly affixed to one of the layers which is immediately adjacent to it, in this case, the sheath 16.

Preferably, the distal end of the counter electrode 14 is fully arranged inside the external dielectric screen 15. Optionally, when at least the distal end 11b of the instrument 11 extends rectilinearly the distance in the direction X separating the distal face of the counter electrode 14 from that of the sheath 16 is between 0.01 and 100 millimetres, and for example, between 1 and 20 millimetres, and is for example, 5 millimetres.

The instrument 11 thus comprises, from the inside to the outside, the following successive layers:
the power supply electrode 12,
the internal dielectric screen 13,
the spacer layer 17,
the sheath 16,
the counter electrode 14,
the external dielectric screen 15.

The instrument 11 comprises, only in this case, these six layers.

The different layers all extend coaxially between those and in the direction X.

Thus, the internal dielectric screen 13 extends coaxially to the power supply electrode 12 directly around it.

The spacer layer 17 extends coaxially to the internal dielectric screen 13 and directly around it.

The sheath 16 extends coaxially to the spacer layer 17 and directly around it.

The counter electrode 14 extends coaxially to the sheath 16 directly around it.

The external dielectric screen 15 extends coaxially to the counter electrode 14 directly around it.

In this case, it is also retained that the power supply electrode 12 dielectric screen 13 are integral with one another.

In this case, it is also retained that the sheath 16, the counter electrode 14 and the external dielectric screen 15 are integral with one another.

The assembly formed of the power supply electrode 12 and of the internal dielectric screen 13 can however slide along the assembly formed by the sheath 16, the counter electrode 14 and the external dielectric screen 15.

Optionally, when at least the distal end of the instrument 11 extends rectilinearly:
the distal faces of the sheath 16 and of the external dielectric screen 15 are at the same level (in the direction X),
and/or
the distal faces of the external dielectric screen 15 and of the counter electrode 14 are not at the same level, as the length of the counter electrode 14 is preferably less than that of the external dielectric screen 15 (in the direction X),
and/or
the distal faces of the sheath 16 and of the counter electrode 14 are not at the same level, as the length of the counter electrode 14 is preferably less than that of the sheath 16 (in the direction X).

In the present case, at the distal end 11b of the instrument 11, the power supply electrode 12 is arranged inside the internal dielectric screen 13, the distal end of which is itself closed and itself arranged inside the sheath 16 and/or the counter electrode 14 and/or the external dielectric screen 15. However, the distal face of the counter electrode 14 is preferably recessed with respect to the distal faces of the sheath 16 and of the external dielectric screen 15; these two latter being preferably in one same plane.

In the present case, at the proximal end 11a of the instrument 11, the power supply electrode 12 is arranged inside the internal dielectric screen 13, the proximal end of which is itself closed and itself arranged inside the sheath 16 and/or the counter electrode 14 and/or the external dielectric screen 15. It is also noted that the proximal faces of at least the sheath 16, the counter electrode 14 and the external dielectric screen 15 are preferably all at the same level. Consequently, the external dielectric screen 15 and the sheath 16 in this case have the same length (in the direction X).

The total length of the sheath 16 and/or of the counter electrode 14 and/or of the external dielectric screen 15 (in the direction X) is in this case of between 0.05 millimetres and 5 metres, and preferably of between 1 and 3 metres and preferably of between 1.5 and 2.5 metres and is preferably of 2 metres. In the present case, the sheath 16 and the external dielectric screen 15 have the same length (in the direction X).

Optionally, the tool 10 and/or the associated device 1 comprises at least one cable 20 for guiding the tool 10.

Such a guide cable 20 facilitates the movement of the tool 10 in the body 100 of the patient, in particular if this relates to making the tool 10 pass into cavities of a very small diameter.

The guide cable 20 extends, in this case, through the whole instrument 11 to open out outside of the two ends of the instrument 11. Preferably, the guide cable 20 extends into the instrument 11 in the space delimited between the internal dielectric screen 13 and the sheath 16. If the spacer layer 17 is present, then the guide cable passes through the gaps of this spacer layer 17. The guide cable 20 passes, for example, between two consecutive segments 19 of one same ring 18 and this, for all the rings 18 of the spacer layer 17.

It is moreover noted that the gas supply source 4, like the gas discharge system 6 are also connected and delimited between the internal dielectric screen 13 and the sheath 16.

In the case where this space is very large (for example, going up to a radial distance between the internal dielectric screen 13 and the sheath 16 of 10 millimetres), it is possible to make other elements that the guide cable 20 has pass into the space delimited by the spacer layer 17, such as for example, a stent.

In service, an electrical current can pass into the power supply electrode 12, which will lead to, by potential difference between the power supply electrode 12 and the counter electrode 14, the generation of a plasma inside the instrument 11 and/or outside of the instrument 11 according to the operating mode of the chosen device.

It is therefore possible to treat a zone by moving the distal end 11b of the instrument 11 closer to said zone. Preferably, the zone to be treated is exposed to the plasma for a time interval of between 0.01 seconds and 2 hours and preferably, between 10 seconds and 30 minutes, and preferably, between 1 and 10 minutes.

Moreover, the following can be defined:
a first gap between the distal face of the power supply electrode 12 and the distal face of the internal dielectric screen 13 (in the direction X), called internal gap or L1, and
a second gap between the distal face of the internal dielectric screen 13 and the zone to be treated (in the direction X), called external gap.

The first gap, which corresponds to the distance L1, has already been treated beforehand.

The second gap is itself between, for example, a few hundred microns and several centimetres. By adjusting, for example, the external gap and/or the time of exposure to the plasma, it is thus possible to adapt the treatment, in particular, according to the sought treatment aim.

The device 1 and, in particular, the tool 10 thus described enables a targeted application of a plasma on a patient.

It is further noted that the plasma generated is advantageously a “cold plasma”, i.e., a plasma outside of the thermodynamic equilibrium where the temperature of electrons is a lot greater than that of ions, itself greater than those of neutral species (atoms and molecules). The temperature of this cold plasma is in line with the body of the patient. This plasma is generated at atmospheric pressure and does not consequently require any particular enclosure (for example, a vacuum enclosure). The inventors have thus been able to develop a prototype generating a plasma, the temperature of the gas of which is less than 40 degrees Celsius, thus facilitating its direct application to the human body.

Furthermore, the device 1 and, in particular, the tool 10 thus described has an electrical isolation making its use very safe for the user, like for the patient. Finally, the tool 10 does not impact or barely impacts the tissues surrounding the zone to be treated.

In this first embodiment, the plasma generated is called “volumic”, because it can extend into the volume separating the internal dielectric screen 13 from the sheath 16, volume defined by the spacer layer 17. It is retained that the plasma generated is fully contained in this volume (concerning the instrument), the plasma moreover being able to be propagated outside of the instrument 11 to the zone to be treated.

As an option, the tool 10 and/or the device 1 comprises at least one cap arranged at the distal end of the instrument 11.

In reference to FIG. 2a, without a cap, the plasma is mainly propagated in the axial extension of the distal end 11b of the instrument 1.

With a cap, it is however possible to modify this propagation.

FIG. 3 illustrates different possible cap shapes.

The cap is preferably made of metal and, for example, made of copper and/or aluminium.

The cap is physically in contact with the counter electrode 14. Thus, the electrical potential of the cap corresponds to that of the counter electrode and can therefore be at the potential of the earth or at a floating potential.

The cap can be arranged at the distal end 11b of the instrument or, on the contrary, extend it. In this case, the cap extends coaxially to the instrument 11 and therefore to the direction X.

The distal end of the cap can have a flat or rounded distal face. Optionally, the distal face is rounded. For example, the distal face is shaped in a dome and in particular, in a half-dome. The distal face is optionally shaped in a geodesic dome.

The cap can comprise at least one meshed zone and/or at least one solid zone (i.e. with no holes nor orifices other than that optionally present to let the guide cable pass through).

Preferably, the meshed zone has a regular mesh.

The mesh can be a one-dimensional network (for example, formed only of rings or only of branches), or a two-dimensional network (the mesh thus being formed of an intersection between rings and branches).

In both cases, the rings are preferably coaxial to the direction X. The branches are preferably coaxial to the direction X.

In both cases, the rings extend preferably at regular intervals from one another and/or coaxially to one another and/or are identical to one another.

In both cases, the branches extend preferably at regular intervals from one another and/or parallel to one another and/or are identical to one another.

The distance separating two consecutive branches is, for example, of between 1 micrometre and 5 millimetres and preferably, of between 50 and 750 micrometres, and is preferably of 250 micrometres. The distance separating two consecutive rings is, for example, of between 1 micrometre and 5 millimetres and, preferably, of between 50 micrometres and 750 micrometres, and for example, of 250 micrometres.

Thanks to the cap, it is thus possible to adapt the shape of the plasma plume to the targeted application type. It is, in particular, possible to radially project the plasma to directly treat the internal wall of the channel 102 and/or that of the cavity 101 in n which the endoscope 2 is introduced.

According to a first option A, the cap 21a is arranged simply at the distal end 11b of the instrument 11. At least the portion of the cap 21a outside of the instrument 11 is meshed and is moreover shaped in a dome.

According to a second option B, the cap 21b is arranged to extend the distal end 11b of the instrument 11. The portion of the cap 21b outside of the instrument 11 comprises a solid, cylindrical section which is extended from a completely meshed distal end and moreover shaped in a dome.

According to a third option C, the cap 21c is arranged to extend the distal end 11b of the instrument 11. The portion of the cap 21c outside of the instrument 11 comprises a meshed cylindrical section which is extended from a solid distal end and moreover shaped in a dome.

According to a fourth option D, the cap 21d is arranged to extend the distal end 11b of the instrument 11. The portion of the cap 21d outside of the instrument 11 comprises a meshed cylindrical section which is extended from a meshed distal end and is moreover shaped in a dome.

In reference to FIGS. 4a and 4b, a tool 10 according to a second embodiment of the invention will now be described.

The instrument 11 of said tool 10 comprises at least from the inside to the outside, at least four layers:
a power supply electrode 12,
an internal dielectric screen 13,
a counter electrode 14,
a sheath 16.

The power supply electrode 12 is therefore a polarised electrode. The power supply electrode 12 is made of an electrically conductive material and, for example, made of a metal and for example, copper- and/or aluminium-based.

Preferably, the power supply electrode 12 is a single wire. The power supply electrode 12 is thus very structurally simple.

The external diameter of the power supply electrode 12 is, for example, of between 2 micrometres and 10 millimetres and, for example, of between 2 micrometres and 2 millimetres, and for example, of between 50 and 350 micrometres and, for example, of between 100 and 150 micrometres and is, for example, of 100 micrometres.

It is noted that the power supply electrode 12 is directly connected to the electrical energy power supply source 4. Consequently, its electrical potential is not floating and only depends on electrical features of the electrical energy power supply source 4 which are moreover known and fully controlled. Consequently, the power supply electrode 12 is an electrode, the value of which is always known (and therefore non-floating).

Thus, the risk of seeing the plasma travel to the thermal arc system is limited. The instrument is therefore very safe to use for the practitioner, as for the patient.

The internal dielectric screen 13 is, for example:
shaped in a tube which is closed at its two axial ends, or
corresponds to a coating directly affixed to the power supply electrode 12, the coating thus also covering the axial ends of the power supply electrode 12 (the internal dielectric screen 13 thus not having a specific shape, as it is not affixed to the power supply electrode 12).

In this way, the power supply electrode 12 is fully arranged inside the internal dielectric screen 13: the plasma cannot therefore come back into contact with said power supply electrode 12 in the same way as the zone to be treated or the immediate environment of said zone (tissue, biological fluid, etc.).

This makes the instrument very safe to use for the practitioner, as for the patient.

Optionally, when at least the distal end 11b of the instrument 11 extends rectilinearly:
the distance in the direction X separating the distal face of the power supply electrode 12 from that of the internal dielectric screen 13 is of between 0 (the value 0 being excluded from the interval) and 20 millimetres, and preferably is of between 0.5 and 5 millimetres, and preferably of between 0.8 and 3 millimetres, and is, for example, of 1 millimetre or 2.5 millimetres,
and/or
the distance in the direction X separating the distal face of the internal dielectric screen 13 from the distal end of the sheath 16 is of between 0 millimetres and 1 metre, and preferably is of between 2 and 10 millimetres, and preferably of between 4 and 7 millimetres, and is for example, of 5 millimetres.

The distance in the direction X separating the distal face of the internal dielectric screen 13 from that of the sheath 16 can be equal to zero, for example and in a non-limiting manner, for dermatological applications.

The “power supply electrode 12 and internal dielectric screen 13” assembly not being integral in the present case with the counter electrode 14 and with the sheath 16, the distance separating them can easily be modified during an intervention by the practitioner, as needed.

The internal dielectric screen 13 is naturally made of a dielectric material.

The internal dielectric screen 13 is, for example, made of a natural r artificial rubber. The internal dielectric screen 13 is, for example, made of plastic material (polyvinylidene fluoride, polytetrafluoroethylene, perfluoroalkoxy, etc.).

The external diameter of the internal dielectric screen 13 is, for example, of between 0.01 and 30 millimetres, and for example, of between 2 and 3.6 millimetres, and, for example, of between 2.5 and 3 millimetres and is, for example, of 2.8 millimetres (being understood that this diameter is moreover necessarily greater than that of the power supply electrode 12).

The counter electrode 14 is preferably a grounding electrode. In a variant, the counter electrode 14 is a floating electrode.

The counter electrode 14 is, for example, made of metal. The counter electrode 14 is optionally copper- and/or aluminium-based.

The counter electrode 14 is shaped in a tube which is open at its two axial ends, or corresponds to a coating layer directly affixed to one of the layers which is immediately adjacent to it, in this case, the internal dielectric screen 13 without covering the distal ends of said layer (the counter electrode 14 thus not having a specific shape, as it is not affixed to said lower layer).

The external diameter of the counter electrode 14 is, for example, of between 0.3 and 36 millimetres, and for example of between 1.4 and 3.6 millimetres and, for example, of between 2.5 and 3.5 millimetres and is, for example, of 3 millimetres (being understood that this diameter is moreover necessarily greater than that of the lower layer, which is directly adjacent to it).

In reference to FIG. 4a, if the counter electrode 14 is shaped in a cylinder of revolution, the plasma is mainly propagated in the axial extension of the distal end 11b of the instrument 11.

The distal end of the counter electrode 14 can however be shaped differently, in order to modify this propagation as can be seen in FIG. 5.

The distal end of the counter electrode 14 can have a flat or rounded distal face. Preferably, the distal face is rounded. For example, the distal face is shaped in a dome and in particular, in a half-dome. The distal face is optionally shaped in a geodesic dome.

The distal end of the counter electrode 14 can comprise at least one meshed zone and/or at least one solid zone (i.e. with no hole nor orifice other than that optionally present to let a guide cable 20 pass through, which will be described below).

Preferably, the meshed zone has a regular mesh.

The mesh can be a one-dimensional network, formed for example of branches, or two-dimensional (the mesh thus being formed of an intersection between rings and branches).

In both cases, the rings are preferably coaxial to the direction X. The branches are preferably coaxial to the direction X.

In both cases, the rings preferably extend at regular intervals from one another and/or coaxially to one another and/or are identical to one another.

In both cases, the branches preferably extend at regular intervals from one another and/or parallel to one another and/or are identical to one another.

The distance separating two consecutive branches is, for example, of between 1 micrometre and 5 millimetres and preferably of between 50 and 750 micrometres, and is preferably of 250 micrometres. The distance separating two consecutive rings is, for example, of between 1 micrometre and 5 millimetres and preferably of between 50 micrometres and 750 micrometres and, for example, of 250 micrometres.

Thanks to the distal end of the counter electrode 14, it is thus possible to adapt the shape of the plasma plume to the targeted application type. It is, in particular, possible to radially project the plasma to directly treat the internal wall of the channel 102 and/or those of the cavity 101, wherein the endoscope 2 is introduced.

For example, the distal end of the counter electrode 14 has a meshed zone, the length of which (in the direction X) is preferably of between 1 millimetre and 2 metres, and preferably of between 1 and 10 centimetres and is preferably of 5 centimetres. The meshed zone starts, in this case, at the distal face.

The length of the counter electrode 14 (in the direction X) outside of said meshed zone is preferably of between 0.1 metre and 50 metres, and is preferably between 1.5 and 2 metres and is preferably of 1.95 metres.

According to a first option A, the distal face is dome-shaped. The whole distal end is meshed according to a two-dimensional mesh.

According to a second option B, the distal face is dome-shaped. The whole distal end is meshed according to a two-dimensional mesh apart from the distal face, which is solid.

According to a third option C, the distal face is flat. The whole distal end is meshed according to a two-dimensional mesh, apart from the distal face, which is solid.

According to a fourth option D, the distal face is dome-shaped. The whole distal end is meshed according to a one-dimensional mesh.

According to a fifth option E, the distal face is flat. The whole distal end is meshed according to a one-dimensional mesh apart from the distal face, which is solid.

According to a sixth option F, the distal face is flat. The whole distal end is meshed according to a one-dimensional mesh, including the distal face.

The sheath 16 is, for example, shaped in a tube which is open at its two axial ends.

Thus, the sheath 16 forms a carrier structure for the instrument.

The sheath 16 is preferably made of a dielectric material.

The sheath 16 is, for example, made of a natural or artificial rubber. The sheath 16 is, for example, made of plastic material (polyvinylidene fluoride, polytetrafluoroethylene, perfluoroalkoxy, etc.).

The sheath 16 is preferably transparent.

The sheath 16 is, for example, between 0.9 and 56 millimetres, and for example, between 1.9 and 8 millimetres and, for example, between 3.5 and 4.5 millimetres, and is for example, 4 millimetres (being understood that this diameter is moreover necessarily greater than that of the counter electrode 14).

Preferably, the instrument 11 comprises at least one additional layer arranged between the internal dielectric screen 13 and the sheath 16.

The additional layer is, for example, a spacer layer 17.

The spacer layer 17 is, in this case, arranged between the counter electrode 14 and the sheath 16.

The external diameter of the spacer layer 17 is, for example, between 0.8 and 46 millimetres, and for example, between 1.6 and 7 millimetres, and for example, between 0.3 and 0.4 millimetres, and is for example, 3.8 millimetres (being understood that this diameter is moreover greater than that of the lower layer which is directly adjacent to it).

The spacer layer 17 is, in this case, composed of a plurality of spacer rings which are independent from one another. However, each of these rings enables the same gap between the counter electrode 14 and the sheath 16, such that they together form a general spacer layer 17.

Preferably, the rings are arranged at regular interval around the counter electrode 14 in the direction X. The distance (in the direction X) separating two consecutive rings is therefore the same between two pairs of different rings.

The distance separating two consecutive rings (in the direction X) is, for example, less than 15 centimetres and preferably less than 10 centimetres.

At least one of the rings is made of a dielectric material. At least one of the rings is, for example, made of a natural or artificial rubber. At least one of the rings is, for example, made of plastic material (polyvinylidene fluoride, polytetrafluoroethylene, perfluoroalkoxy, etc.). Preferably, the different rings are identical to one another, such that the description below of one of the rings is also applicable to the other rings.

The ring is, in this case, composed at least of two elements which are independent from one another. In the present case, the ring is composed of four elements which are independent from one another.

The ring is mainly shaped in a ring, each element forming a segment of this ring. The four elements are preferably distributed regularly circumferentially around the counter electrode 14. Each element is arranged substantially at 90 degrees from each of the two elements of the same ring surrounding it.

Preferably, each ring is oriented in the same way vis-à-vis the counter electrode 14. Consequently, a segment of a ring is necessarily aligned (in the direction X) with another segment of each of the other rings.

Each element being identical, the description below of one of the segments is also applicable to the other segments. The length of the segment (in the direction X) is preferably of between 0.2 millimetres and 100 millimetres, and preferably of between 2 and 20 millimetres, and preferably of between 3 and 6 millimetres, and is preferably of 5 millimetres.

Moreover, the thickness of the segment (in a radial direction) is preferably of between 10 micrometres and 10 millimetres, and preferably of between 0.1 and 1 millimetre, and is preferably of 0.5 millimetre.

Each segment can extend so as to have an external periphery forming an angle sector of between 35 and 50 degrees and, for example, being 45 degrees.

The instrument 11 thus comprises from the inside to the outside, the following successive layers:
the power supply electrode 12,
the internal dielectric screen 13,
the counter electrode 14,
the spacer layer 17,
the sheath 16.

The instrument 11 comprises, only in this case, these five layers.

The different layers all extend coaxially between those and in the direction X.

Thus, the internal dielectric screen 13 extends coaxially to the power supply electrode 12 directly around it.

The counter electrode 14 extends coaxially to the internal dielectric screen 13 by being coupled to it.

The spacer layer 17 extends coaxially to the counter electrode 14 by being coupled to it.

The sheath 16 extends coaxially to the spacer layer 17 directly around it.

It is also retained that the power supply electrode 12 and the first dielectric screen 13 and the counter electrode 14 are integral with one another.

At the distal end 11b of the instrument 11, the power supply electrode 12 is arranged inside the internal dielectric screen 13, the distal end of which is closed and itself arranged inside the sheath 16 and/or the counter electrode 14. However, the distal faces of the sheath 16 and of the counter electrode 14 are preferably not at the same level (in the direction X) and open (preferably, the counter electrode 14 is indeed arranged inside the sheath 16).

It is also noted that the proximal faces of at least the sheath 16 and the counter electrode 14 are preferably not at the same level, preferably, the counter electrode 14 is indeed arranged inside the sheath 16. Consequently, these two layers do not have the same length (in the direction X). The total length of the sheath 16 and/or of the counter electrode 14 (in the direction X) is, in this case, of between 0.05 millimetre and 5 metres, and preferably of between 1 and 3 metres and preferably of between 1.5 and 2.5 metres, and is preferably of 2 metres. In the present case, the sheath 16 and the counter electrode 14 do not have the same length (in the direction X).

Optionally, the tool 10 and/or the associated device 1 comprises at least one cable 20 for guiding the tool 10.

Such a guide cable 20 facilitates the movement of the tool 10 in the body 100 of the patient, in particular if this relates to making the tool 10 pass into cavities of a very small diameter.

The guide cable 20 extends, in this case, through the whole instrument 11 to open out outside of the two ends of the instrument 11. Preferably, the guide cable 20 extends into the instrument 11 in the space delimited between the counter electrode 14 and the sheath 16. If the spacer layer 17 is present, then the guide cable passes through the gaps of this spacer layer 17.

It is moreover noted that the gas supply source 4, like the gas discharge system 6, are also connected in the space delimited between the counter electrode 14 and the sheath 16.

In the case where this space is very large (for example, by going up to a radial distance between the counter electrode 14 and the sheath 16 of 10 millimetres), it is possible to make other elements that the guide cable 20 has pass into the space delimited by the spacer layer 17, such as for example, a stent.

In service, an electrical current is passed into the power supply electrode 12, which will lead, by potential difference between the power supply electrode 12 and the counter electrode 14, to the generation of a plasma inside the instrument 11 and/or outside 4 the instrument 11 according to the operating mode of the device 1 chosen.

It is therefore possible to treat a zone by moving the distal end 11b of the instrument 11 closer to said zone.

Preferably, the zone to be treated is exposed to the plasma for a time interval of between 0.01 second and 2 hours, and preferably of between 10 seconds and 30 minutes, and preferably of between 1 and 10 minutes.

The following can moreover be defined:
a first gap between the distal face of the power supply electrode 11 and the distal face of the internal dielectric screen 13 (in the direction X), called internal gap, and
a second gap between the distal face of the internal dielectric screen 13 and the zone to be treated (in the direction X), called external gap.

The first gap has already been treated beforehand. The second gap is itself between, for example, a few hundred microns and several centimetres.

By adjusting, for example, the second gap and/or the time of exposure to the plasma, it is thus possible to adapt the treatment, in particular, according to the sought treatment aim.

The device 1 and, in particular, the tool 10 thus described enables a targeted application of a plasma on a patient.

It is further noted that the plasma generated is advantageously a “cold plasma”, i.e. a plasma outside of the thermodynamic equilibrium where the temperature of electrons is a lot greater than that of ions, itself greater than those of neutral species (atoms and molecules). The temperature of this cold plasma is in line with the body of the patient. This plasma is generated at atmospheric pressure and consequently requires no particular enclosure (for example, a vacuum enclosure). The inventors have thus been able to develop a prototype generating a plasma, the temperature of the gas of which is less than 40 degrees Celsius, thus facilitating its direct application to the human body.

Furthermore, the device 1, and in particular, the tool 10 thus describes has an electrical isolation making its use very safe for the user, like for the patient. Finally, the tool 10 does not impact or barely impacts the tissues surrounding the zone to be treated.

In this second embodiment, the plasma generated is called “surface” plasma, because it can extend into the space separating the power supply electrode 12 of the counter electrode 14, space of small dimensions, because the counter electrode 14 is directly coupled to the internal dielectric screen 13, itself directly affixed to the power supply electrode 12. The plasma is mainly only propagated along the counter electrode 14. It is retained that the plasma generated is fully contained in this space (concerning the instrument), the plasma moreover being able to be propagated outside of the instrument 11 towards the zone to be treated.

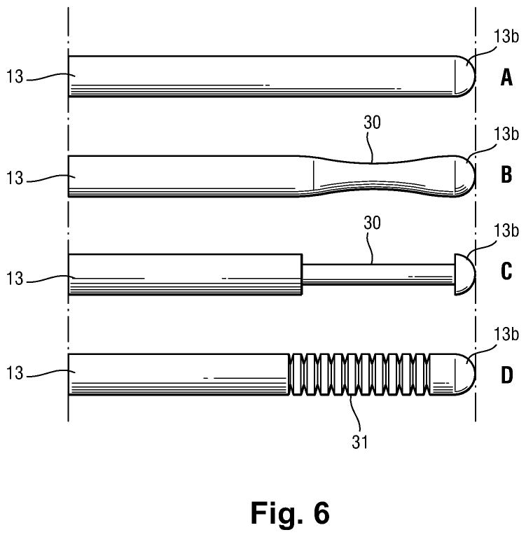

In reference to FIG. 6, it is possible to adapt other parameters of the device 1, and in particular, of the tool 10, and even more specifically, of the instrument 11 to the targeted treatment.

For example, at least one of the layers can have at least one distal end shaped in another way than in a straight cylinder (when at least the distal end of the instrument extends in the direction X).

In the different FIGS. 1 to 5, the internal dielectric screen 13 is always shaped in a straight cylinder.

The internal dielectric screen 13 can however be shaped in another way.

According to a first variant A, the distal end 13b of the internal dielectric screen 13 is rounded. The distal end 13b of the internal dielectric screen 13 thus substantially forms a dome at least at its distal face. The rest of the internal dielectric screen 13 is shaped in a straight cylinder.

According to a second variant B, the internal dielectric screen 13 successively has, from its proximal end to its distal end:
a proximal section, including the proximal end, shaped in a straight cylinder,
a rounded distal end 13b (for example, substantially forming a dome, at least at its distal face),
a connection section 30 between the proximal section and the rounded distal end, the connection section 30 forming a displacement.

The internal dielectric screen 13 thus locally has a section narrowing between the proximal section and the distal end 13b at its connection section 30. This narrowing is formed by a curve provided in the internal dielectric screen 13. The narrowing is thus shaped in a groove of significant length (in the direction X).

The maximum diameter of the distal end 13b is substantially equal to that of the proximal section. According to a third variant C, the internal dielectric screen 13 successively has, from its proximal end to its distal end 13b:
a proximal section, including the proximal end, shaped in a straight cylinder,
a rounded distal end 13b (for example, substantially forming a dome at least at its distal face), a connection section 30 between the proximal section and the rounded distal end 13b, the connection section 31 also being shaped in a straight cylinder of diameter less than that of the proximal section.

The internal dielectric screen 13 thus locally has a section narrowing between the proximal section and the distal end 13b at the connection section 31.

However, different from the second variant B, the narrowing 30 is formed at its two longitudinal ends (in the direction X) by two straight connectors, one with the proximal section, the other with the rounded distal end 13b. The narrowing is thus shaped in a kerf of significant length (in the direction X).

The maximum diameter of the distal end 13b is substantially equal to that of the proximal section.

The distal end 13b is shaped, in this case, in a half-dome, the base of which is connected to the connection section 30.

According to a fourth variant D, the internal dielectric screen 13, successively has, from its proximal end to its distal end:
a proximal section, including the proximal end, shaped in a straight cylinder,
a distal 13b rounded end (for example, substantially forming a dome at least at its distal face),
a connection section 31 between the proximal section and the rounded distal end 13b, the connection section 31 comprising a succession of recesses and/or kerfs along the direction X.

Different from the second variant B and from the third variant C, the recesses and/or the kerfs are of small dimensions, but there are more of them. The recesses and/or the kerfs are preferably all identical to one another and/or arranged at regular intervals along the direction X.

Also, according to another variant, the internal dielectric screen 13 successively has, from its proximal end to its distal end 13b:
a proximal section, including the proximal end, shaped in a straight cylinder,
a rounded distal end 13b (for example, substantially forming a dome at least at its distal face),
a connection section between the proximal section and the rounded distal end, the connection section being shaped in a screw thread and therefore being externally threaded.

Naturally, the invention is not limited to the embodiments described, and variants can be provided without moving away from the scope of the invention such as defined by the claims.

As indicated, the device can comprise other elements than what has been indicated, like for example, one or more other tools inserted into the applicator (biopsy tool, camera-type observation tool, illumination tool, etc.), in addition to the tool described, dedicated to generating a plasma.

The spacer layer and/or the external dielectric screen can be avoided.

The spacer layer can be different from what has been indicated. For example, the spacer layer can be of one single part or be formed of a different number of elements to what has been indicated.

The spacer layer can comprise at least two spacer rings different from one another and/or spaced apart differently from another pair of spacer rings. At least one of the rings can be of one single part or be formed of a different number of elements to what has been indicated. The ring can thus be formed of two elements connected to one another, for example, by a ring of diameter less than the external diameter of the elements.

The cap can also be avoided.

The distal end of the instrument can also be protected, for example, by covering, at least partially, said end of a tip made of a hydrophobic material, in order to limit the risk of a fluid penetrating inside the instrument. The cap and/or the distal end of the counter electrode can directly form this tip by being made of a hydrophobic material. In case there is a tip and a cap, one can be equally placed outside of the other one. Naturally, if there is a tip and a cap, it will be ensured that the tip always enables the passage of the plasma to the outside.

Although each of the layers described (excluding spacer layer) can be formed by depositing a coating on the immediately adjacent lower and/or upper layer or by a pipe-type tube, at least one of said layers can be formed in another way. For example, at least one of the layers can be formed by winding a wire around the immediately adjacent lower layer. The wire will naturally be wound very tightly in order to limit, as much as possible, a space between the turns and thus form a uniform layer. For example, the counter electrode can be formed in this way (for the two embodiments described) by winding a metal wire.

The two embodiments described can naturally be mixed. For example, the distal end of the counter electrode of the first embodiment can be shaped like in the second embodiment and/or the instrument of the second embodiment can comprise a cap like in the first embodiment.

If a distal face is not flat, the distance taken between said face and another point will be implicitly considered as being taken at the most distal point from said face and the other point.

Although, in this case, the tool is shaped to be able to be used with an endoscope, the tool can be shaped in another way. For example, the tool will comprise at least one needle. Typically, the needle will have an external diameter of between 1 and 5 mm, and an internal diameter of between 20 μm and 3 mm. For example, the needle will be made of metal.

The needle will thus be connected to a distal end of the tool. The needle can thus be connected to the counter electrode 14 and, in particular, to its distal end.

This needle will make it possible to use the tool, no longer according to the endoscopic approach as described above, but according to the percutaneous approach.

This needle will be shaped to enable the plasma to reach the zone to be treated. For example, the needle will be hollow.

1. A tool for generating a plasma, the tool comprising at least one instrument wherein the instrument comprises at least, from the inside to the outside:
a power supply electrode,
an internal dielectric screen,
a sheath,
the instrument further comprising at least one counter electrode arranged externally to the internal dielectric screen.

2. The tool according to claim 1, wherein the power supply electrode is a metal wire.

3. The tool according to claim 1, wherein the power supply electrode is polarised and the counter electrode is a grounding electrode.

4. The tool according to claim 1, wherein the instrument comprises at least one additional layer.

5. The tool according to claim 4, wherein the additional layer is chosen from among:
an external dielectric screen and/or
a spacer layer.

6. The tool according to claim 5, wherein the additional layer is a spacer layer which is arranged on the internal dielectric screen or on the counter electrode.

7. The tool according to claim 5, wherein the instrument comprises two additional layers, namely an external dielectric screen and a spacer layer, the spacer layer being arranged on the internal dielectric screen and the external dielectric screen on the counter electrode.

8. The tool according to claim 5, wherein the spacer layer is composed of a plurality of spacer rings which are independent from one another.

9. The tool according to claim 1, comprising at least one cap arranged at the distal end of the instrument to guide, in service, a passage of the plasma from the inside of the instrument to the outside of the instrument.

10. The tool according to claim 9, wherein the cap is an integral part of the counter electrode thus forming the distal end of said counter electrode.

11. The tool according to claim 9, wherein the cap has at least one hydrophobic surface zone.

12. The tool according to claim 1, wherein at least the distal end of the counter electrode is shaped in a cylinder.

13. The tool according to claim 1, comprising a hollow needle arranged on a distal part of the tool.

14. A medical treatment device comprising an applicator, wherein at least one conduit is provided, a tool according to claim 1, being arranged, such that at least its instrument extends into said conduit.

15. The medical treatment method implemented using a device according to claim 14, comprising the steps of moving the applicator closer to a zone to be treated and of generating a plasma by the tool in order to subject said zone to be treated to said plasma.
